(19) United States
(12) Patent Application Publication
(10) Pub. No.: US 2018/0133012 A1
(43) Pub. Date: May 17, 2018

(54) IMPLANTABLE DEVICE FOR IMPROVING OR TREATING A HEART VALVE INSUFFICIENCY

(71) Applicants: Universitat Duisburg-Essen, Essen (DE); coramaze technologies GmbH, Munich (DE)

(72) Inventors: Niklas Maximilian NATHE, Wolfratshausen (DE); Till NEUMANN, Bochum (DE); Torsten SCHEUERMANN, Munich (DE)

(21) Appl. No.: 15/573,454

(22) PCT Filed: May 10, 2016
(86) PCT No.: PCT/EP2016/000758

§ 371 Date: Nov. 10, 2017

(30) Foreign Application Priority Data
May 12, 2015 (DE) 10 2015 005 934.3

(51) Int. Cl. A61F 2/24

(52) U.S. Cl. CPC A61F 2/2478; A61F 2/2463; A61F 2/2454; A61F 2/2412; A61F 2220/0075; A61F 2/246; A61F 2230/0004; A61F 2220/0016; A61F 2210/0014

(57) ABSTRACT

The invention relates to an implantable device for improving or treating a heart valve insufficiency, comprising a closure body (1) and at least one anchoring element fastened thereto, by means of which the closure body (1) can be fastened in the heart, preferably in the atrium of the heart, in which the anchoring element comprises a plurality of contact strips (2) extending between the closure body (1) and a shared connecting element (3) which is spaced from the closure body (1), wherein, due to a shape change of the contact strips (2), the connecting element (3) can be brought into a first and a second position each spaced from the closure body, wherein, in the first position, the connecting element (3) is spaced farther apart from the closure body (1) than in the second position, and in the second position, the connecting element (3) is arranged between the contact strips (2).

The invention relates to an implantable device for improving or treating a heart valve insufficiency, comprising a closure body and at least one anchoring element fastened thereto, by means of which the closure body can be fastened in the heart, preferably in the atrium of the heart.

Implantable devices of this type are known from the prior art, for example, from the application DE 10 2012 010 798 A1 of the same inventor, and they are used if, in a patient, the heart valves do not close sufficiently during the usual heart activity, and, as a result, incorrect flows between ventricle and atrium of the heart arise.

An implantable device of the type mentioned at the start is then implanted in the heart of the patient, so that the closure body is arranged in the passage area of the heart valve and the atrioventricular valves of the heart valve thus are in contact at the time of closure with the closure bodies, and, accordingly, the inserted closure body closes a gap area which would otherwise remain.

By means of the closure body of such an implantable device, an improved closure of the heart valve is thus reestablished, and the above-mentioned incorrect flows are prevented.

The fastening of such a device by means of the anchoring element fastened thereto can occur here in such a manner that the anchoring element on the closure body allows an atraumatic fastening in the heart, particularly due to the fact that, for the purpose of the fastening, the anchoring element is in contact with the inner wall of the heart, without penetrating the heart wall.

For example, an anchoring element can be formed for this purpose by a strip or by a plurality of strips or strip sections, wherein a respective strip or strip section extends away from the closure body optionally with ramifications. As a result, a basket-like structure is formed, which can contact the heart wall, particularly the wall of the atrium, from inside, especially if strips forming a loop are led back to the closure body.

Since, in this fastening method, it is desirable that contact with the heart wall, particularly in the atrium, occurs from inside, an anchoring element of this type, in the implanted state after fastening, has a size corresponding at least substantially to the volume of the atrium, preferably slightly larger in order to achieve a resulting contact pressure.

Accordingly, in such devices it is problematic to introduce an anchoring element with such a large cross section into the heart, a procedure which at this time is performed essentially surgically by opening the heart in a corresponding complicated and risky operation.

On this backdrop, an aim of the invention is to provide a device of the generic type mentioned at the start which can be implanted in a minimally invasive manner, preferably transfemorally, in the body of a patient. It is preferable here to create a self-expanding device.

This aim is achieved according to the invention in that the anchoring element comprises several contact strips extending between the closure body and a shared connecting element which is spaced from the closure body, wherein, due to a shape change of the contact strips, the connecting element can be brought into a first and a second position each spaced from the closure body, wherein, in the first position, the connecting element is spaced farther apart from the closure body than in the second position, and in the second position, the connecting element is arranged between the contact strips.

The invention preferably provides here for the device to comprise precisely a single anchoring element formed by the contact strips and lying on only one side of the closure body, in particular an anchoring element arranged on the atrium side.

A contact strip is preferably understood to refer to each strip- or wire-shaped element which extends at least in certain areas away from the closure body for the formation of the anchoring element and leads into the connecting element, optionally including ramifications or cross connections.

The two positions here should preferably be selected in such a manner that the connecting element in both positions lies on an imaginary line which coincides with the longitudinal extent direction of the closure body, preferably of a closure body whose cross section can be changed perpendicularly to the longitudinal extent, for example, in that the closure body has at least one cavity which can be filled with a fluid, wherein the at least one cavity has a constant volume after the filling, that is to say the volume thereof does not automatically change subsequently; in particular, during the heart phases, there is constancy of volume in spite of the changing pressure conditions.

Although the invention is preferably usable in connection with such a closure body, it can be used in principle with any closure body known in the prior art or yet to be developed in the future.

This type of anchoring body can also be used not only with closure bodies for treating a heart valve insufficiency, but also, in principle, with any implant which is to be anchored in the heart.

The essential core idea of the invention is based on the fact that the anchoring element can assume two shapes, which is achieved by the change in shape of the contact strips, wherein, for the two shapes, the position of the connecting element is different.

Therefore, a connecting element is understood to refer to the element which connects the ends of the contact strips extending away from the closure body. In a possible design, these ends of the contact strips can be the initially open, free ends which are connected by the connecting element.

Since the contact strips have a fixed length, assuming the first position, in which the connecting element is spaced farther from the closure body than in the second position, means that the contact strips are elongated, preferably maximally elongated, and in closer contact with one another than is the case in the second position. In particular, in this position, the contact strips can touch one another. In particular, the elongation can be such that the contact strip, at least in certain sections, in each case extend linearly between closure body and connecting element.

In this first position, the entire device can be introduced, for example, into a catheter, in order to be brought into the heart through said catheter, for example, transfemorally. For this purpose, in the first position, the maximum cross section of the entire device is smaller than the inner cross section of the catheter.

In the second position, the connecting element is not only moved closer to the closure body in comparison to the first position, as a result of which the contact strips move farther apart from one another in a direction perpendicular to the longitudinal extent of the device, but also is positioned so close that it enters an area between the contact strips, and thereby preferably lies on the imaginary line mentioned at the start, which coincides with the longitudinal extent direction.

The arrangement of the connecting element between the contact strips preferably means that, in a view of a cross section perpendicular to the connection direction between ventricle and atrium, i.e., perpendicular to the longitudinal extent of the device, the connecting element positioned between the closure body and the atrium-side end area of the anchoring element preferably lies in the middle between the contact strips; the contact strips thus surround this connection element.

Preferably, an arrangement of the connecting element between the contact strips in the second position, moreover or in other words, is understood to mean that, in a direction perpendicular to the line mentioned at the start, in particular to a central axis line leading through the closure body and the connecting element, areas of several contact strips are located laterally next to the connecting element, in particular areas of several strips are arranged around this line/central axis line.

However, in the first position, the arrangement is preferably such that there are no areas at all of one or more contact strips in the above-mentioned direction perpendicular to the line/central axis line, laterally next to the connecting element, in particular over the total axial length thereof. The contact strips which are connected to the connecting element are preferably connected to said connecting element on the axial front side of the connecting element pointing toward the closure body.

In a preferred manner, in the second position, the end of the connecting element pointing away from the closure body as a result no longer forms the extreme end of the entire device, but instead lies in front of it, i.e., closer to the closure body. In this manner it is possible to achieve that the connecting element itself does not come in contact with the heart inner wall, but preferably only the contact strips come in contact, in particular with the outer sides thereof.

Preferably, in the second position, the contact strips are pushed apart from one another, preferably maximally, to the extent that the contact strips overall form a basket structure which peripherally defines a cross section which corresponds at least to the cross section of the atrium used for the fastening or is greater than said cross section.

In the second position assumed, the mentioned basket structure forms an inversion pointing in the direction toward the interior of the basket structure. This inversion is preferably arranged symmetrically around the mentioned central axis line and moreover preferably forms an annular space at least in certain areas, which is formed around the connecting element and which moreover is preferably open toward the outside. “Toward the outside” here means means from the connecting element when viewed in the direction pointing away from the closure body. The outwardly pointing opening of the inversion has an opening plane which is, in particular, imagined to be tangentially in contact with the basket structure and to lie perpendicularly to the central axis line, wherein the connecting element is arranged between this plane and the closure element in the second position.

The design of the device can be selected here in such a manner that, in the second position, the contact strips in each case have in certain areas an extent directed backward toward the closure body and/or are shaped convexly at the end of the anchoring element maximally spaced from the closure body, and/or have an S-shaped extent in certain areas, particularly adjoining the connecting element. In particular, the vertex areas of the S-shaped extent can form the areas directed backward toward the closure body.

For the formation of a stabilizing anchoring element, it is possible to provide that the contact strips are connected to one another by struts or ramifications.

Moreover, the device can have a design provided with an equal number of contact strip sections extending away from the closure body and of contact strip sections leading into the connecting element.

In particular, the group of the closure body-side contact strip sections can here be offset relative to the group of the connecting element-side contact strip sections by an angle around the extension direction (the imaginary line mentioned at the start).

It is possible to provide that each contact strip section of one group splits into at least two contact strip branches and is connected via these branches to at least two contact strip sections of the other group.

Here, the contact strip branches of adjacent branched contact strip sections can intersect; in particular, they can be connected at the intersection points.

Preferably, it is possible to provide in all the possible embodiments that the contact strips are connected in each case at the end pointing toward the closure body to an inner base body of the connecting element around which the surfaces of the closure element interacting with the heart valves are arranged. Preferably, these interacting surfaces are the wall area(s) of at least one cavity which can be filled with a fluid and which is arranged around a base body.

Here, the term contact strips, in the sense of the above-mentioned designs, also subsumes the term contact strip sections, particularly those on the closure body side.

The base body can be, for example, a tubular element, around which at least one fillable cavity is arranged, as mentioned at the start.

In a simple design, the base body can be formed by a rigid tube, in particular a tube which is surrounded by the at least one cavity. A preferred design can also provide that the base body is formed as an element which can be bent out of the linear extension thereof, in particular an element which can be relaxed back into the linear extension due to inner spring forces.

Such a base body is thus preferably flexible in an automatically resettable manner.

In a design as, in particular, a metal tubular body, the base body can be wound at least in certain sections to form a spring, for example, made of a wire with round cross section or else of a strip with angular cross section.

A bendable base body can also be produced from an originally rigid tube, by introducing a plurality of cuts into the lateral surface of the tubular base body. Cuts can be located preferably perpendicularly to the longitudinal extent and are spaced equidistantly preferably in longitudinal direction.

Cuts that succeed one another in longitudinal direction can also be produced alternately in different directions, the meaning of this being preferably that cuts are made in different angular positions relative to an angle viewed in peripheral direction. The angular positions can be equidistant, for example, cuts are located preferably at 0 degree, 90 degrees, 180 degrees and 270 degrees.

In a respective longitudinal position, in each case two cuts can also face one another, each ending before the center of the tube, for example, a pair of opposite cuts at 0 and 180 degrees as well as a pair at 90 and 270 degrees. The pairs are thus spaced in longitudinal direction, preferably equidistantly.

An automatically resetting bendability of a tubular base body can also be implemented by means of one or more meandering cuts in the lateral surface of the base body. If there are several cuts, they can be interlaced. Such a meandering cut can comprise cut sections extending alternately in longitudinal direction and transversely, preferably perpendicularly thereto.

In a preferred development, the invention can provide that at least the connecting element, the contact strips and an inner base body of the closure body, for example, of the above-described closure body, are designed to form a single piece with one another, in particular they are cut, preferably laser cut, from a length of tube.

Preferably, the contact strips here too again include the closure body-side and connecting element-side contact strip sections and all the contact strip branches connecting said contact strip sections or branching from them.

In this single-piece design, most of the device can thus be produced from a semifinished product, for example, a tube, particularly made of a shape memory material.

In all the designs, the invention can provide that the connecting element can be transferred by a force pointing away from the closure body, from the second position into the first position, particularly against a spring tension applied to the contact strips. This force can be exerted on the connecting element, for example, by means of a sliding catheter which pushes the connecting element away from the closure body. Here, the sliding catheter can be led, for example, through the closure body and abut against the connecting element.

A design can also provide that the connecting element can be moved from the first position into the second position by means of forces produced by a temperature change from a first temperature to a second temperature in the contact strips formed from a shape memory material, wherein, in particular, at the first and second temperatures, the corresponding first and second positions each form a stable position of the contact strips.

For example, the shape of the contact strips assumed in the first position can form a stable position at a first lower temperature, and the shape of the second position can form a stable position at a temperature which is elevated in comparison to the first temperature. For example, the device can be implanted cooled, for example, under continuous cooling during the advance in a catheter, for example, a catheter exposed to a cooling fluid.

After the implantation, the device heats up and is converted into the shape in which the connecting element assumes the second position. Due to the assuming of the second position, the anchoring element then fulfills the anchoring function thereof.

For the purpose of the implantation, the connecting element can comprise an inner open cross section, in particular it can be designed in the form of a ring or tube sleeve. A guide wire can be pushed through the connecting element, for example, after it has been led through the closure body, particularly through the base body thereof.

The sliding catheter already been mentioned above can be led on the guide wire up to the connecting element which is sleeve-shaped, for example, and abuts against said connecting element, since it cannot penetrate through the connecting element. A pushing of the sliding catheter has the effect that the connecting element, which is sleeve-shaped, in particular, and which has been pushed, pulls behind it the rest of the device, particularly the closure body, near to the contact strips.

An embodiment of the invention is explained in reference to the following figures.

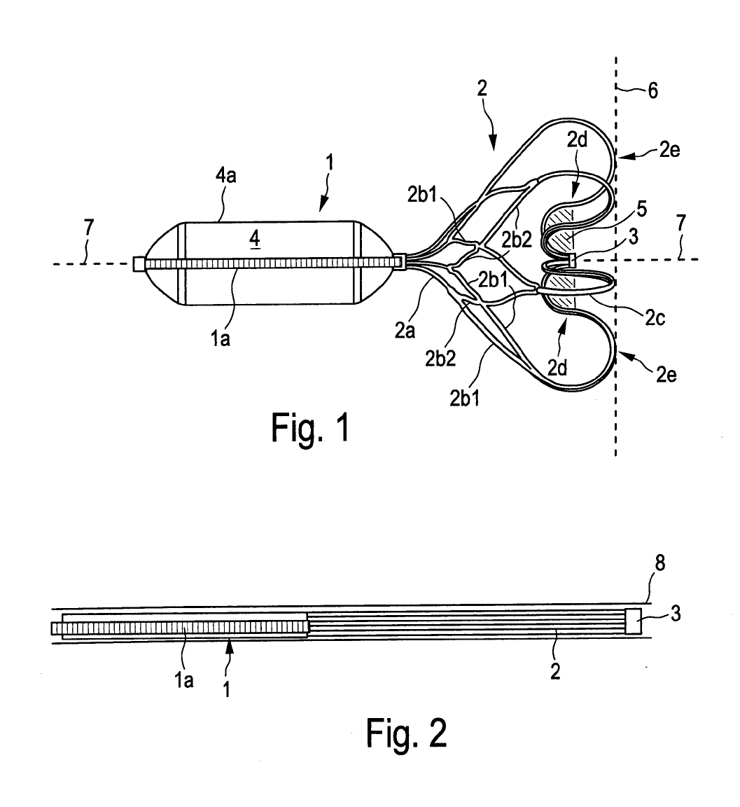

FIG. 1 shows a side view of a device according to the invention with a closure body 1, here a fillable closure body of the type mentioned above, with at least one cavity 4 which is arranged around a base body 1a. The cavity 4 is delimited outwardly by the wall 4a. The closure body 1 is represented here on the left side.

Overall, the contact strips 2 extending away from the closure body 1 on the right side form a kind of basket- or cage-like structure and, with this structure they form the anchoring element according to the invention, here in the expanded state after the implantation.

The contact strips 2 are divided into closure body-side contact strip sections 2a which extend away from the closure body 1 or the base body 1a and which split in each case into two contact strip branches 2b1 (to the right) and 2b2 (to the left). Two contact strip branches 2b1, 2b2 of different contact strip sections 2a are again joined in a connecting body-side contact strip section 2c.

FIG. 1 shows that here the connecting element 3 has assumed a position close to the closure body, in which it is arranged between the contact strips 2. According to the above general description, this is the second position. The areas of the contact strips 2 or of the contact strip sections 2c, which adjoin the connecting element 3, are bent to the shape of an S. In the areas 2d marked here, particularly the areas which form the vertex points of the S-shaped bending, the contact strips 2 or the contact strip sections 2c extend backward toward the closure body 1.

The contact strips 2, here the connecting element-side contact strip sections 2c thereof, form an area 2e which is convex toward the heart wall and which comes to abut in a fastening manner on the heart inner wall of the atrium. Here, the connecting element 3 is located closer to the closure body 1 than the convex end areas 2e.

In this construction, the annular space 5, represented with cross hatching here, which is open in the direction pointing away from the closure body 1, extends around the connecting element 3. This annular space 5 here forms the base area of an inversion of the basket- or cage-shaped anchoring element formed by the contact strips 2. The opening of the inversion, which points away from the closure body 1, has an opening plane 6, visualized by the dashed line. This opening plane 6 extends perpendicularly with respect to the center axis line 7 extending through the closure body 1 and the connecting element 3 and is in tangential contact with the contact strips 2. The connecting element 3 lies in the second position shown here, that is to say in the fully expanded state of the anchoring element, between the closure body 1 and said opening plane 6. As a result, one can see that, when the anchoring has taken place, the connecting element 3 cannot come in contact with the heart muscle tissue.

FIG. 2 shows the device in a state in which the connecting element 3 is in a position in which it is spaced farther from the closure body 1 than in the second position. The spacing here is at a maximum, so that the contact strips 2 or the respective contact strip sections are elongated maximally and extend substantially linearly. Here, as in FIG. 1, one can see that, on the axial front side/front surface of the connecting element 3 pointing towards the closure body, the contact strips 2 lead into said connecting element or are connected thereto.

Due to the elongation of the contact strips 2, they lie close to one another, so that the device has a smaller overall cross section perpendicular to the direction of extent than the inner diameter of a catheter 8 in which the device can be brought to the implantation site.

1. An implantable device for improving or treating a heart valve insufficiency, comprising a closure body (1) and at least one anchoring element fastened thereto, by means of which the closure body (1) can be fastened in the heart, preferably in the atrium of the heart, characterized in that the anchoring element comprises a plurality of contact strips (2) extending between the closure body (1) and a shared connecting element (3) which is spaced from the closure body (1), wherein, due to a shape change of the contact strips (2), the connecting element (3) can be brought into a first and a second position each spaced from the closure body, wherein, in the first position, the connecting element (3) is spaced farther apart from the closure body (1) than in the second position, and in the second position, the connecting element (3) is arranged between the contact strips (2).

2. The device according to claim 1, characterized in that, in the second position, the contact strips (2) in each case
a. comprise in certain areas an extent (2d) directed backward toward the closure body (1), and/or
b. are shaped convexly at the end (2e) of the anchoring element maximally spaced from the closure body (1), and/or
c. have in certain areas an S-shaped extent, particularly adjoining the connecting element (3).

3. The device according to any one of the preceding claims, characterized in that the contact strips (2) are connected to one another by struts.

4. The device according to any one of the preceding claims, characterized in that it comprises precisely a single anchoring element formed by the contact strips and lying on only one side of the closure body (1), in particular an anchoring element arranged on the atrium side.

5. The device according to any one of the preceding claims, characterized in that the contact strips (2) are connected in each case at the end pointing toward the closure body (1) to an inner base body (1a) of the closure body (1), around which the surfaces (4a) of the closure body (1) that interact with the heart valves are arranged.

6. The device according to any one of the preceding claims, characterized in that at least the connecting element (3), the contact strips (2) and an inner base body (1a) of the closure body (1) are designed to form a single piece, and in particular are cut, preferably laser cut, from a length of tube.

7. The device according to any one of the preceding claims, characterized in that the connecting element (3) can be transferred by a force pointing away from the closure body (1), from the second position into the first position, in particular against a spring tension applied to the contact strips (2).

8. The device according to any one of the preceding claims, characterized in that the connecting element (3) can be transferred from the first position into the second position by forces generated due to temperature change in the contact strips (2) formed from a shape memory material, in particular wherein, at the respective temperatures, the first position and the second position both form a stable position of the contact strips.

9. The device according to any one of the preceding claims, characterized in that the connecting element (3) has an inner open cross section designed in particular as a ring or tube sleeve.
